(19) United States
(12) Patent Application Publication
(10) Pub. No.: US 2024/0318124 A1
(43) Pub. Date: Sep. 26, 2024

(54) PREDICTIVE CELL-BASED FED-BATCH PROCESS

(71) Applicant: JANSSEN BIOTECH, INC., HORSHAM, PA (US)

(72) Inventors: JAMES VINCENT PRICE, MALVERN, PA (US); COLLEEN FRIDLEY, MALVERN, PA (US); STEVEN SAVAGE, MALVERN, PA (US)

(21) Appl. No.: 18/579,249

(22) PCT Filed: Jul. 13, 2022
(86) PCT No.: PCT/US22/36913

§ 371 Date: Jan. 12, 2024

Related U.S. Application Data
(60) Provisional application No. 63/221,183, filed on Jul. 13, 2021.
(60) Provisional application No. 63/221,197, filed on Jul. 13, 2021.
(60) Provisional application No. 63/221,174, filed on Jul. 13, 2021.

(51) Int. Cl. C12M 1/36; C12M 1/00; C12M 1/06; C12M 1/34; C12N 5/071

(52) U.S. Cl. CPC C12M 41/48; C12M 27/02; C12M 29/06; C12M 41/30; C12N 5/0602

(57) ABSTRACT

Methods and systems related to delivery of complex feed nutrients based on a number of cells are presented herein. A method of controlling a nutrient feed, a method of developing a feeding schedule, and a nutrient feed control system are presented herein. Volume of feed per day can be proportional to a predicted change in integrated viable cells (IVS) from the present feeding day to the next feeding day. A per cell factor (PCF) can be determined by determining a normalized feed per cell value for a time interval of a preliminary fed-batch bioreactor run in which the feed consumed is approximately equal to the feed provided. The volume of feed per day can be set equal to a product of the PCF and change in IVS from the present feeding day to the next feeding day.

### CROSS REFERENCE TO RELATED APPLICATIONS

The present application claims benefit of U.S. Provisional Patent Application No. 63/221,174 filed Jul. 13, 2021, U.S. Provisional Patent Application No. 63/221,183 filed Jul. 13, 2021, and U.S. Provisional Patent Application No. 63/221,197 filed Jul. 13, 2021. The entire contents of each of which are hereby incorporated by reference herein.

### BACKGROUND

Cell culture productivity relies on optimization of culture medium management to enable high cell densities and productivity. Nutrient feeding is an important parameter for process optimization in fed-batch cell feeding processes. High cell density processes can require substantial amounts of nutrients, and the daily requirements can vary with cell type and/or cell density. One known complex nutrient feeding strategy in fed-batch processes involves providing feed to a bioreactor based on a fixed percentage of bioreactor weight or bioreactor volume. Alternative cell culture processes which can result in an increase in cell culture productivity are desirable.

### SUMMARY

Improved materials and methods for the prediction of daily nutrient target requirements for a given cell line is addressed by the present disclosure. One aspect of the disclosed technology relates to a method of controlling a nutrient feed in a cell culture process, specifically, delivery of complex feed nutrients that is based on number of cells. Volume of feed per day can be proportional to a predicted change in integrated viable cells (IVC) from the present feeding day to the next feeding day. A per cell factor (PCF) is developed herein. The volume of feed per day can be set equal to a product of the PCF and change in IVC from the present feeding day to the next feeding day. The PCF can be further refined to vary over time during a fed-batch process.

An aspect of the disclosed technology relates to a method of executing a cell-based fed-batch bioreactor run. A quantity of feed may be provided to a bioreactor based at least in part on an estimated feed consumption rate (qₛ) and based at least in part on a predicted change in integrated viable cell (IVC) number, the predicted IVC number being from a present feeding interval to a future feeding interval.

The change in IVC number can be predicted as follows: an IVC number of a present feeding interval can be determined, an IVC number of a previous feeding interval can be determined, a growth rate can be estimated based on the viable cell density of the present feeding interval and the viable cell density of the previous feeding interval, an IVC number for a following feeding interval can be based at least in part on the estimated growth rate, and the change in IVC can be set equal to the IVC number for the following feeding interval minus the IVC number for the present feeding interval.

Providing the quantity of feed to a bioreactor can be based at least in part on a Per Cell Factor (PCF). The PCF can be proportionate to the estimated feed consumption rate (qₛ).

The PCF can vary over time during the cell-based fed-batch bioreactor run. In one embodiment, the PCF does not increase over time during the cell-based fed-batch bioreactor run. In one embodiment, the PCF is approximately equal to 0.002 g/cell×day for culture days 2 through 3 of the cell-based fed-batch bioreactor run; the PCF is approximately equal to 0.00175 g/cell×day for culture days 4 through 6 of the cell-based fed-batch bioreactor run; the PCF is approximately equal to 0.0015 g/cell×day for culture days 7 through 8 of the cell-based fed-batch bioreactor run; the PCF is approximately equal to 0.0012 g/cell×day for culture days 9 through 10 of the cell-based fed-batch bioreactor run; and the PCF is approximately equal to 0.0007 g/cell×day for culture days 11 through 13 of the cell-based fed-batch bioreactor run.

The quantity of feed can be provided to the bioreactor such that for each feed interval during the fed-bath bioreactor run, an amount of metabolite added to the bioreactor during each respective feed interval run is approximately equal to an amount of metabolite consumed within the bioreactor during each respective feed interval.

The cell-based fed-batch bioreactor run can be completed such that between about 20% and about 50% of a volume of the bioreactor consists of feed at the completion of the cell-based fed-batch bioreactor run.

Providing the quantity of feed can include providing a complex nutrient feed comprising at least one of, but not limited to: alanine, arginine, asparagine, aspartic acid, cysteine, cystine, glutamic acid, glutamine, glycine, histidine, isoleucine, leucine, lysine, methionine, phenylalanine, proline, hydroxy-L-proline, serine, threonine, tryptophan, tyrosine, valine, amino acid di- and tri-peptides, B vitamins, carbohydrates, lipids, antioxidants, growth factors and trace elements

The quantity of feed can be provided to the bioreactor in an initial stage having a first plurality of feeding intervals and a subsequent final stage having a second plurality of feeding intervals. The quantity of feed can increase from one feeding interval to a subsequent feeding interval for each of the first plurality of feeding intervals of the initial stage. The quantity of feed can decrease from one feeding interval to a subsequent feeding interval for each of the second plurality of feeding intervals of the final stage.

The cell-based fed-batch bioreactor run can be completed such that a viable cell density in the bioreactor is approximately 3 to approximately 5 times a resulting viable cell density of a volume-based fed-batch bioreactor run executed under identical conditions, but for feeding strategy, as the cell-based fed-batch bioreactor run.

The estimated feed consumption rate (qₛ) and IVC can each be based on a reduced-scale study of the cell-based fed-batch bioreactor run. The estimated feed consumption rate (qₛ) and IVC can each be based on about a 0.25 L study of the cell-based fed-batch bioreactor run.

Another aspect of the disclosed technology relates to a method of developing a cell-based fed-batch feeding schedule. A fed-batch bioreactor run can be executed on a cell line in which feeds are added at a regular time interval. For each regular time interval, a normalized feed per cell value can be calculated as follows: for each regular time interval calculate an interval feed per cell value that is a feed quantity fed at the respective time interval divided by a change in an integrated viable cell (IVC) number from a previous time interval to the respective time interval, determine a maximum interval feed per cell value of the calculated interval feed per cell values, and for each regular time interval set the normalized feed per cell value equal to the interval feed per cell value for the respective time interval divided by the maximum interval feed per cell value. For each regular time interval, a normalized IVC number can be calculated as follows: for each regular time interval estimate a daily IVC number based at least in part on an estimated IVC number of the previous time interval and a change in viable cells from the previous time interval to the respective time interval, determine a maximum daily IVC number of the estimated daily IVC numbers, and for each regular time interval set the normalized IVC number to the estimated daily IVC number divided by the maximum daily IVC number. A balanced feed time interval can be selected from the regular time intervals such that the normalized feed per cell value is approximately equal to the normalized IVC number for the balanced feed time interval. A per cell factor (PCF) can be set equal to the interval feed per cell value of the balanced feed time interval. The cell-based fed-batch feeding schedule can be developed such that feed amounts are determined for each feeding interval and each respective feed amount is based at least in part on the PCF and is proportional to a predicted change in IVC number from a present feeding interval to a future feeding interval.

At least four fed-batch bioreactor runs can be executed on the cell line in which feeds are added at the regular time interval. For each regular time interval of each of the fed-batch bioreactor runs, the normalized feed per cell value can be calculated. For each regular time interval of each of the fed-batch bioreactor runs, the normalized IVC number can be calculated. For each fed-batch bioreactor run, the balanced feed time interval can be selected. For each fed-batch bioreactor run, the PCF can be set equal to the respective interval feed per cell value of the respective balanced feed time interval. An average PCF can be determined that is an average of the PCFs of the at least four fed-batch bioreactor runs. The cell-based fed-batch feeding schedule can be developed such that feed amounts are determined for each feeding interval and each respective feed amount is equal to the average PCF multiplied by the predicted change in IVC number.

A first cell-based fed-batch bioreactor run can be executed according to the fed-batch feeding schedule. An updated PCF can be determined based at least in part on said PCF and based at least in part on the first cell-based fed-batch bioreactor run. The updated PCF can result in an updated fed-batch feeding schedule that results in a decreased total amount of feed added at an end of a second cell-based fed-batch bioreactor run compared to the total amount of feed added at an end of the first cell-based fed-batch bioreactor run. Reducing the amount of feed added while meeting cell nutritional requirements will lower the culture osmolality providing a more optimal environment for cell growth and recombinant protein production.

The first cell-based fed-batch bioreactor run can be executed according to the fed-batch feeding schedule. A variable PCF and a variable fed-batch feeding schedule can be determined based at least in part on said PCF and based at least in part on the first cell-based fed-batch bioreactor run.

The PCF can vary over time during the cell-based fed-batch bioreactor run. In one embodiment, the PCF does not increase over time during the cell-based fed-batch bioreactor run. In one embodiment, the PCF is approximately equal to 0.002 mL/cell×day for culture days 2 through 3 of the cell-based fed-batch bioreactor run; the PCF is approximately equal to 0.00175 mL/cell×day for culture days 4 through 6 of the cell-based fed-batch bioreactor run; the PCF is approximately equal to 0.0015 mL/cell×day for culture days 7 through 8 of the cell-based fed-batch bioreactor run; the PCF is approximately equal to 0.0012 mL/cell×day for culture days 9 through 10 of the cell-based fed-batch bioreactor run; and the PCF is approximately equal to 0.0007 mL/cell×day for culture days 11 through 13 of the cell-based fed-batch bioreactor run.

The cell-based fed-batch feeding schedule is developed at a reduced scale to provide feed targets that are transferrable across scales. The cell-based fed-batch feeding schedule is developed at about a 0.25 L scale and is transferrable across fed-batch production processes utilizing about 250 liter (L) and about 1,000 L bioreactors respectively.

Another aspect of the disclosed technology relates to a nutrient feed control system. The nutrient feed control system can include a viable cell density measurement system, a future viable cell density prediction system, a per cell factor (PCF) system, and a feed calculation system. The viable cell density measurement system can be configured to determine density of viable cells in a production reactor. The future viable cell density prediction system can be configured to receive the viable cell density measurement and can be configured to estimate a density of viable cells in a future feed time interval based at least in part on the viable cell density measurement. The PCF system can be configured to provide a PCF value. The feed calculation system can be configured to calculate an amount of feed to provide to the production reactor based at least in part on the density of viable cells in the future feed time interval and based at least in part on the PCF value.

The PCF system can further be configured to determine a balanced feed time interval at which metabolites consumed in the bioreactor are approximately equal to metabolites provided to the bioreactor during the balanced feed time interval. The PCF system can further be configured to determine a feed per cell value for the balanced feed time interval. The PCF system can further be configured to determine the PCF value based at least in part on the feed per cell value for the balanced feed time interval.

In one embodiment, the nutrient feed control system can further include a processor and non-transitory computer-readable medium in communication with the processor with instructions thereon that can be executed by the processor. The instructions can cause the processor to calculate the amount of feed to provide to the production reactor. Additionally, or alternatively, the instructions can cause the processor to determine the PCF value.

The feed calculation system can further be configured to scale the amount of feed to provide to a larger production reactor of one or more larger scale fed-batch production processes. The feed calculation system can further be configured to scale the amount of feed to provide to the larger production reactor based on a 0.25 L scale process. The feed calculation system can be configured to scale the amount of feed to provide to 250 liter (L) and a 1,000 L bioreactors during respective larger scale red-batch production processes.

### BRIEF DESCRIPTION OF THE DRAWINGS

Reference will now be made to the accompanying drawings, which are not necessarily drawn to scale, and which are incorporated into and constitute a portion of this disclosure, illustrate various implementations and aspects of the disclosed technology, and, together with the description, explain the principles of the disclosed technology.

FIG. 1 is a plot representing an example cell-based fed-batch feeding schedule that may be used to implement one or more embodiments of the present disclosure in comparison to a volume-based fed-batch feeding schedule known in the art.

FIG. 2 is a plot representing quantity of metabolite added per feeding interval of respective fed-batch bioreactor runs executing the cell-based fed-batch feeding schedule and the volume-based fed-batch feeding schedule illustrated in FIG. 1.

FIG. 3 is a plot of normalized feed per cell and normalized IVC for determining a PCF that may be used to implement one or more embodiments of the present disclosure.

FIG. 4 is a plot of quantity of feed added per day of fed-batch bioreactor runs including volume-based fed-batch bioreactor runs and iterations of cell-based fed-batch bioreactor runs in which the PCF value is updated for each subsequent cell-based fed-batch bioreactor run according to aspects of the present disclosure.

FIG. 5 is a plot of cell viability percentage for two cell lines of a volume-based fed-batch bioreactor run as known in the art and a cell-based fed-batch bioreactor run according to aspects of the present disclosure.

FIG. 6 is a plot of viable cell density (measured in 106 viable cells (vc)/mL) for the two cell lines of the volume-based fed-batch bioreactor run and the cell-based fed-batch bioreactor run presented in FIG. 5.

FIG. 7 is a plot of relative concentration of arginine (measured in absorbance units (AU)) in the bioreactor during a volume-based fed-batch bioreactor run as known in the art, a first iteration of a cell-based fed-batch bioreactor run according to aspects of the present disclosure, and a final iteration of a cell-based fed-batch bioreactor run according to aspects of the present disclosure.

FIG. 8 is a plot of relative concentration of histidine (measured in absorbance units (AU)) in the bioreactor during a volume-based fed-batch bioreactor run as known in the art, a first iteration of a cell-based fed-batch bioreactor run according to aspects of the present disclosure, and a final iteration of a cell-based fed-batch bioreactor run according to aspects of the present disclosure.

FIG. 9 is a plot of relative concentration of isoleucine (measured in absorbance units (AU)) in the bioreactor during a volume-based fed-batch bioreactor run as known in the art, a first iteration of a cell-based fed-batch bioreactor run according to aspects of the present disclosure, and a final iteration of a cell-based fed-batch bioreactor run according to aspects of the present disclosure.

FIG. 10 is a plot of relative concentration of leucine (measured in absorbance units (AU)) in the bioreactor during a volume-based fed-batch bioreactor run as known in the art, a first iteration of a cell-based fed-batch bioreactor run according to aspects of the present disclosure, and a final iteration of a cell-based fed-batch bioreactor run according to aspects of the present disclosure.

FIG. 11 is a plot of relative concentration of lysine (measured in absorbance units (AU)) in the bioreactor during a volume-based fed-batch bioreactor run as known in the art, a first iteration of a cell-based fed-batch bioreactor run according to aspects of the present disclosure, and a final iteration of a cell-based fed-batch bioreactor run according to aspects of the present disclosure.

FIG. 12 is a plot of relative concentration of methionine (measured in absorbance units (AU)) in the bioreactor during a volume-based fed-batch bioreactor run as known in the art, a first iteration of a cell-based fed-batch bioreactor run according to aspects of the present disclosure, and a final iteration of a cell-based fed-batch bioreactor run according to aspects of the present disclosure.

FIG. 13 is a plot of relative concentration of phenylalanine (measured in absorbance units (AU)) in the bioreactor during a volume-based fed-batch bioreactor run as known in the art, a first iteration of a cell-based fed-batch bioreactor run according to aspects of the present disclosure, and a final iteration of a cell-based fed-batch bioreactor run according to aspects of the present disclosure.

FIG. 14 is a plot of relative concentration of threonine (measured in absorbance units (AU)) in the bioreactor during a volume-based fed-batch bioreactor run as known in the art, a first iteration of a cell-based fed-batch bioreactor run according to aspects of the present disclosure, and a final iteration of a cell-based fed-batch bioreactor run according to aspects of the present disclosure.

FIG. 15 is a plot of relative concentration of tyrosine (measured in absorbance units (AU)) in the bioreactor during a volume-based fed-batch bioreactor run as known in the art, a first iteration of a cell-based fed-batch bioreactor run according to aspects of the present disclosure, and a final iteration of a cell-based fed-batch bioreactor run according to aspects of the present disclosure.

FIG. 16 is a plot of relative concentration of valine (measured in absorbance units (AU)) in the bioreactor during a volume-based fed-batch bioreactor run as known in the art, a first iteration of a cell-based fed-batch bioreactor run according to aspects of the present disclosure, and a final iteration of a cell-based fed-batch bioreactor run according to aspects of the present disclosure.

FIG. 17 is a flow diagram illustrating steps of an example method for developing a cell-based fed-batch feeding strategy according to aspects of the present disclosure.

FIG. 18 is a flow diagram illustrating steps of an example method for executing a cell-based fed-batch bioreactor run according to aspects of the present disclosure.

FIG. 19 is a schematic diagram of an example environment that may be used to implement one or more embodiments of the present disclosure.

FIG. 20 is a schematic diagram of an example environment that may be used to implement one or more embodiments of the present disclosure.

FIG. 21 is a block diagram of a nutrient feed control system that may be used to implement one or more embodiments of the present disclosure.

FIG. 22 is a bar chart showing daily complex feed targets at three different scales for production of monoclonal antibody 1 (MAB1) according to aspects of the present disclosure.

FIG. 23 is a plot of titer values for three different scales for production of MAB1 according to aspects of the present disclosure.

FIG. 24 is a plot of daily measured osmolality for three different scales for production of MAB1 according to aspects of the present disclosure.

FIG. 25 is a bar chart showing daily complex feed targets at three different scales for production of bi-specific antibody 1 (BsAb1) according to aspects of the present disclosure.

FIG. 26 a plot of daily measured osmolality for three different scales for production of BsAb1 according to aspects of the present disclosure.

### DETAILED DESCRIPTION

Some implementations of the disclosed technology will be described more fully with reference to the accompanying drawings. This disclosed technology may, however, be embodied in many different forms and should not be construed as limited to the implementations set forth herein. The components described hereinafter as making up various elements of the disclosed technology are intended to be illustrative and not restrictive. Many suitable components that would perform the same or similar functions as components described herein are intended to be embraced within the scope of the disclosed electronic devices and methods. Such other components not described herein may include, but are not limited to, for example, components developed after development of the disclosed technology.

It is also to be understood that the mention of one or more method steps does not preclude the presence of additional method steps or intervening method steps between those steps expressly identified.

It is to be noted, unless otherwise clear from the context, that the term “a” or “an” entity refers to one or more of that entity; for example, “an amino acid,” is understood to represent one or more proteins. As such, the terms “a” (or “an”), “one or more,” and “at least one” can be used interchangeably herein.

The term “nutrient” may refer to any compound, molecule, or substance used by an organism to live, grow, or otherwise add biomass. Examples of nutrients may include carbohydrate sources (e.g., simple sugars such as glucose, galactose, maltose or fructose, or more complex sugars), amino acids, vitamins (e.g., B group vitamins (e.g., B12), vitamin A vitamin E, riboflavin, thiamine and biotin). In the present invention, one or more nutrients may be utilized as a surrogate molecule to determine the amount of total nutrient media to add to a bioreactor. In some embodiments, the term “nutrient” may refer to simple sugars, vitamins, and amino acids.

The term “amino acid” may refer to any of the twenty standard amino acids, i.e., glycine, alanine, valine, leucine, isoleucine, methionine, proline, phenylalanine, tryptophan, serine, threonine, asparagine, glutamine, tyrosine, cysteine, lysine, arginine, histidine, aspartic acid and glutamic acid, single stereoisomers thereof, and racemic mixtures thereof. The term “amino acid” can also refer to the known non-standard amino acids, e.g., 4-hydroxyproline, hydroxy-proline, s-sulfocysteine, phosphotyrosine, ε-N,N,N-trimethyllysine, 3-methylhistidine, 5-hydroxylysine, O-phosphoserine, γ-carboxyglutamate, ε-N-acetyllysine, ω-N-methylarginine, N-acetylserine, N,N,N-trimethylalanine, N-formylmethionine, γ-aminobutyric acid, histamine, dopamine, thyroxine, citrulline, ornithine, β-cyanoalanine, homocysteine, azaserine, and S-adenosylmethionine. In some embodiments, the amino acid is glutamate, glutamine, lysine, tyrosine or valine. In some embodiments, the amino acid is glutamate or glutamine.

The terms “nutrient media,” “feed media,” “feed,” “total feed,” “complex feed,” and “total nutrient media” may be used interchangeably, and may include a “complete” media used to grow, propagate, and add biomass to a cell line. Nutrient media may be distinguished from a substance or simple media which by itself is not sufficient to grow and propagate a cell line. Thus, for example, glucose or simple sugars by themselves are not nutrient media, since in the absence of other required nutrients, they would not be sufficient to grow and propagate a cell line.

For specific examples presented herein, a density of biological media in a bioreactor is often assumed to be approximately 1.0 g/mL, and therefore grams and mL can be used interchangeably herein.

In one aspect, the present invention teaches a complex nutrient control algorithm to balance a complex nutrient feed in a bioreactor. Delivery of the complex nutrient feed is an essential part of a fed-batch production bioreactor. To support the development of next generation cell culture processes using genetically engineered cell lines, a feeding strategy based on an integrated viable cell (IVC) number has been created. The aim of this strategy is to deliver complex feed nutrients in proportion to the number of viable cells to prevent under- or over-feeding during the process. Example methods presented herein can use per-cell factors (PCFs) and a predictive IVC calculation to generate daily complex feed amounts. The development of a per cell complex feed strategy is presented herein. An example method for determining a cell line specific PCF for use in calculating nutrient feed quantity is presented herein. An example method for predicting IVC of a subsequent feeding cycle is presented herein.

Examples presented herein may, but are not required to, begin with cells and culture media (i.e., basal or base media) on Day 0. On or around Day 2-4 of the process “feeding” the bioreactor can begin, with additional cell culture media (i.e., feed media) being added to the bioreactor. A number of feed media may be added depending on the process, for instance, two or three feed media may be added. The complex/nutrient/total feed media can include a media that has more than 2 main components (typically around 50-60 components). A glucose solution or a high-pH solution, whose two main components are cystine and tyrosine, may also be added to the bioreactor during the process.

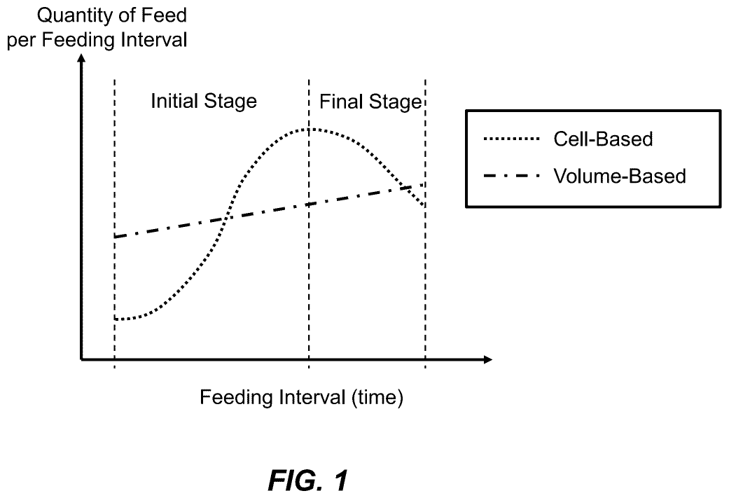

FIG. 1 is a plot representing an example cell-based fed-batch feeding schedule that may be used to implement one or more embodiments of the present disclosure in comparison to a volume-based or weight-based fed-batch feeding schedule known in the art. At the beginning of a fed-batch bioreactor run, number of viable cells is small. The cell-based fed-batch feeding schedule provides feed as a function of number of viable cells, and therefore the feed amount is small in the cell-based approach at the beginning of the bioreactor run. The volume- or weight-based fed-batch feeding schedule provides feed as a function of bioreactor volume or weight, and therefore the feed amount is lower at the beginning of the bioreactor run compared to the remaining time of the bio-reactor run, but is likely much greater than the initial feed of the cell-based fed-batch feeding schedule. In a volume- or weight-based fed-batch feeding schedule, the feed added per feeding interval increases over time during the bio-reactor run as the volume and weight in the bioreactor increases. In a cell-based fed-batch feeding schedule, the quantity of feed per feeding interval likely increases during an initial stage in which the change in viable cells per day increases, and the quantity of feed per feeding interval likely decreases during a final stage in which the change in viable cells per day decreases.

FIG. 1 is intended to provide a general illustration of a likely cell-based fed-batch feeding schedule. It is to be understood that the cell-based approach can be tailored for cell line and components of feed and therefore may deviate from the general illustration in FIG. 1.

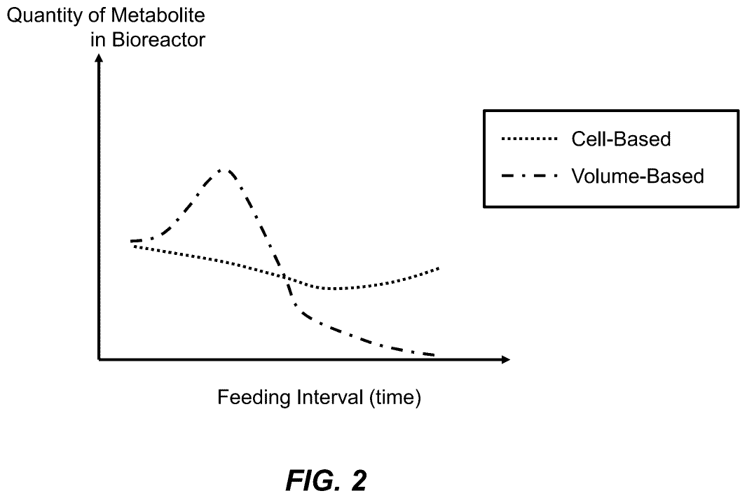

FIG. 2 is a plot representing quantity of metabolite of respective fed-batch bioreactor runs executing the cell-based fed-batch feeding schedule and the volume-based fed-batch feeding schedule illustrated in FIG. 1. In a volume- or weight-based fed-batch feeding schedule, metabolite in the reactor may increase at a beginning of the bioreactor run due to over-feeding and then may decrease after the number of viable cells have increased to the point where they are consuming more nutrients than are being added. An aim of the cell-based fed-batch bioreactor schedule is to maintain quantity of metabolite in the bioreactor at an approximately constant level.

In order to derive an equation to determine a cell-based feeding strategy a few assumptions were taken. Those assumptions are as follows: (1) If the minimal nutrition requirements of each cell is met each day by the complex feed, then there is no net accumulation or depletion of metabolites; and (2) It is assumed that the complex feed formulation is properly balanced and proportioned for a specific cell line expressing monoclonal antibodies.

A mass balance on a metabolite, S, can be written according to Equation (1).

$${S}_{i , d} = {S}_{i , d - 1} + ( {C}_{s} \cdot {V}_{f , d} ) - {Q}_{s} \qquad ( 1 )$$

Where S_{i,d} and S_{i,d-1} are the molar amounts of metabolite, i, in the culture today and yesterday, respectively. The (Cₛ·V_{f,d}) term is the moles added through the complex feed solution each day, and Qₛ is the total moles consumed or produced by the cells. With the assumption that there's no net accumulation or depletion of metabolites the two Sᵢ terms will cancel (i.e., amount of metabolite doesn't change) and Equation (1) can be rearranged according to Equation (2).

$${C}_{s} \cdot {V}_{f , d} = {Q}_{s} \qquad ( 2 )$$

The total consumption term, Qₛ, can then be rewritten in terms of a specific consumption rate multiplied by the change biomass, where X represents viable cells.

$${C}_{s} \cdot {V}_{f , d} = {q}_{s} \cdot \frac{dX}{d t} \qquad ( 3 )$$

The change in biomass can be numerically approximated by using the equation for IVC (Equation (4). Equations (4) and (5) can be combined and solved for V_{f,d} (volume of feed for culture day, d). The resulting equation is Equation (6).

$$\frac{dX}{d t} ≅ IVC = {\sum }_{d = 0}^{d} ( ( \frac{{X}_{v , d} + {X}_{v , d - 1}}{2} ) \cdot ( {t}_{d} - {t}_{d - 1} ) + {IVC}_{d - 1} ) \qquad ( 4 )$$

$${C}_{s} \cdot {V}_{f , d} = {q}_{s} \cdot {\sum }_{d = 0}^{d} ( ( \frac{{X}_{v , d} + {X}_{v , d - 1}}{2} ) \cdot ( {t}_{d} - {t}_{d - 1} ) + {IVC}_{d - 1} ) \qquad ( 5 )$$

$${V}_{f , d} = \frac{{q}_{s}}{{C}_{s}} \cdot IVC \qquad ( 6 )$$

The term,

$$\frac{{q}_{s}}{{C}_{s}} ,$$

is the Per Cell Factor (PCF) in terms of volume per cell per day (Volume/(Cell×Day)). Equation (6) now describes the amount of complex feed to add each culture day based on a predetermined PCF and the change in IVC of the culture.

PCF is a scaling-factor that can be determined to properly scale the complex feed rate according to a cell line's biomass. The PCF can be determined based on a genetically engineered cell line on a small-scale production bioreactor. The PCF can be determined by first calculating a normalized feed per cell value (Equation (7)) and a normalized IVC (Equation (8)). An average PCF can be determined by averaging PCF for multiple bioreactor runs, preferably a minimum of four bioreactor runs per cell line.

$$Normalized Feed per Cell = \frac{{V}_{f 1 , d} {/}_{Δ {IVC}_{d - ( d - 1 )}}}{MAX ( {V}_{f 1 , d} {/}_{Δ {IVC}_{d - ( d - 1 )}} )} \qquad ( 7 )$$

$$Norm . IVC = \frac{{\sum }_{d = 0}^{d} ( ( \frac{{X}_{v , d} + {X}_{v , d - 1}}{2} ) \cdot ( {t}_{d} - {t}_{d - 1} ) + {IVC}_{d - 1} )}{MAX ( {\sum }_{d = 0}^{d} ( ( \frac{{X}_{d} + {X}_{d - 1}}{2} ) \cdot ( {t}_{d} - {t}_{d - 1} ) + {IVC}_{d - 1} ) )} \qquad ( 8 )$$

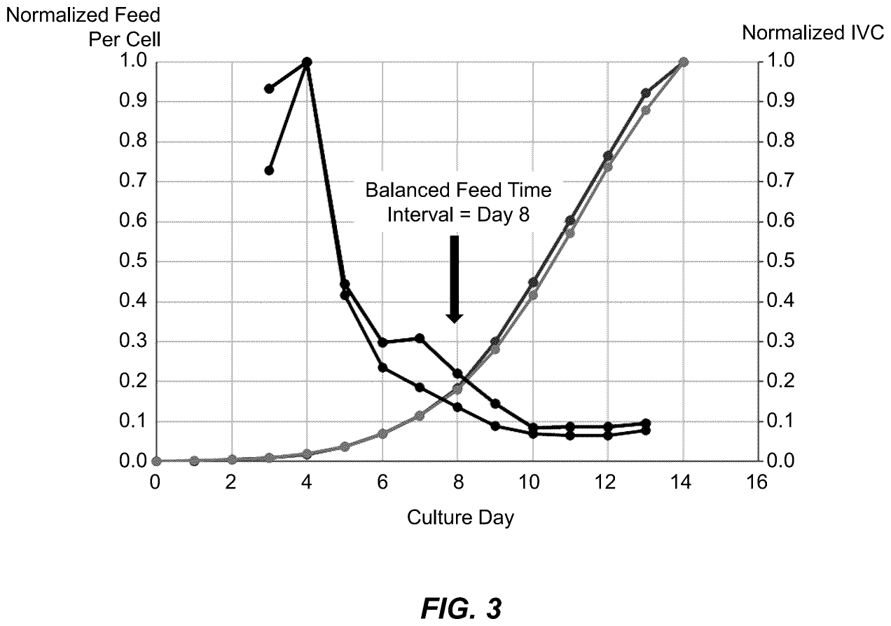

FIG. 3 is a plot of normalized feed per cell and normalized IVC for determining a PCF value. By plotting the time series of these two values on separate y-axes for a bioreactor process, a balanced feed time interval (day) can be determined based on which culture day the normalized feed per cell and the normalized IVC intersect. The intersection of these two lines is assumed to be the culture day where the amount of feed delivered to the bioreactor is proportional to the cell density. In other words, prior to that intersection too much feed is delivered for the current cell density and past that intersection too little feed is delivered for that current cell density.

Once the culture day where feed per cell matches the IVC is determined (balanced feed time interval), the PCF can be determined by taking the average feed per cell value (numerator in Equation (7)) for that culture day. In FIG. 3, the intersection occurs approximately on culture day 8. Therefore, the feed per cell values for culture day 8 were averaged for each of the cell lines. The resulting average PCF are shown in Table 1 for the three GS_CHO cell lines used in the evaluation.

TABLE 1

| | Per Cell Factor | |
|---|---|---|
| Cell Line | (mL/ΔIVC ± SD) | # of Runs |
| Cell Line 1 | 0.0026 ± 0.0007 | 9 |
| Cell Line 2 | 0.0029 ± 0.0001 | 7 |
| Cell Line 3 | 0.0030 ± 0.0006 | 5 |

The values in Table 1 are used for an initial evaluation of this strategy.

The amount of feed to add per day is calculated by multiplying the current day's PCF by the predicted change in IVC (Equation (3), rewritten in a different form in Equation (9).

$$Feed Amount ( g ) = {P CF}_{d} \times ( {IVC}_{d + 1} - {IVC}_{d} ) \qquad ( 9 )$$

The term IVC_{d+1} is the predicted IVC on the following culture day (d+1), which can be calculated as presented in Equation (10).

$${IVC}_{d + 1} = ( \frac{{X}_{v , d + 1} \cdot {V}_{rxr , d + 1} + {X}_{v , d} \cdot ( {V}_{rxr , d} - {V}_{sample , d} )}{2} ) \cdot ( {t}_{d + 1} - {t}_{d} ) + {IVC}_{d} \qquad ( 10 )$$

The term X_{v,d+1} is the predicted cell density, and V_{rxr,d+1} is the predicted reactor volume on the following day as defined in Equations (11) and (12). It is assumed that the feed volumes on the previous day will be similar enough to the current day to allow for a reasonable approximation of the following day's volume prior to feeding. The predicted cell density, X_{v,d+}1, can be calculated by multiplying the current cell density by the exponential of the growth rate over the last day, μ, multiplied by the time between cell density measurements (typically 1 day).

$${X}_{v , d + 1} = {X}_{V , d} \cdot {e}^{μ \cdot t} \qquad ( 11 )$$

$${PCF}_{d} \times ( \frac{{X}_{V , d} \cdot {e}^{μ \cdot t} \cdot ( {V}_{rxr , d} - \frac{{V}_{sample , d}}{1000} + \frac{( {V}_{f 1 , d - 1} + {V}_{f 2 , d - 1} + {V}_{f 3 , d - 1 )} )}{1000} ) + {X}_{v , d} \cdot ( {V}_{rxr , d} - {V}_{sample , d} )}{2} ) \qquad ( 12 )$$

The PCFs generated from the above procedure may be sufficient to develop a cell-based fed-batch feeding schedule. Additionally, or alternatively, the PCFs generated from the above procedure may serve as a starting point for the tuning of the PCF parameter for specific cell lines in High Titer media.

As a demonstration, the PCFs in Table 1 were used to feed 5 L reactors on a per-cell basis.

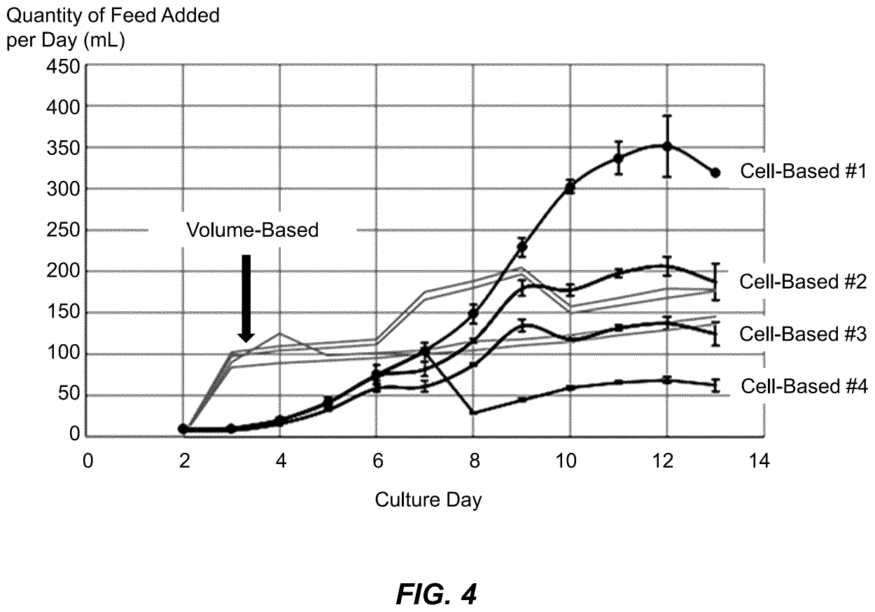

FIG. 4 is a plot of quantity of feed added per day of fed-batch bioreactor runs including volume-based fed-batch bioreactor runs and iterations of cell-based fed-batch bioreactor runs in which the PCF value is updated for each subsequent cell-based fed-batch bioreactor run according to aspects of the present disclosure.

The resulting feed amounts towards the end of the first iteration of a cell-based fed-batch bioreactor run (Cell-Based #1) were above 10% of the daily reactor volume (>300 mL) resulting in high osmolality and limiting process performance. Over a series of experiments (Cell-Based #2, Cell-Based #3, and Cell-Based #4), the PCF were tuned to reduce osmolality accumulation while maintaining productivity and product quality. The final, recommended PCF for GS-CHO cell lines are shown in Table 2.

TABLE 2

| | Per Cell Factor (PCF) |
|---|---|
| Culture Day | (mL/cell*day) |
| 2-3 | 0.002 |
| 4-6 | 0.00175 |
| 7-8 | 0.0015 |
| 9-10 | 0.0012 |
| 11-13 | 0.0007 |

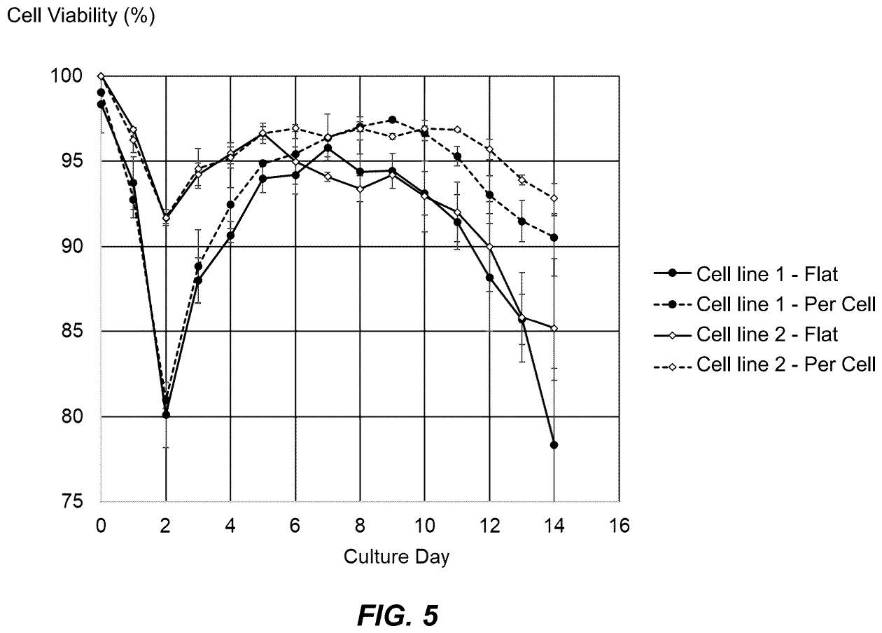

FIG. 5 is a plot of cell viability percentage for two cell lines (Cell Line 1 represented in the plot by black circles and Cell Line 2 represented in the plot by white diamonds) of a weight-based fed-batch bioreactor run (Flat, solid line) as known in the art and a cell-based fed-batch bioreactor run (Per Cell, dashed) according to aspects of the present disclosure.

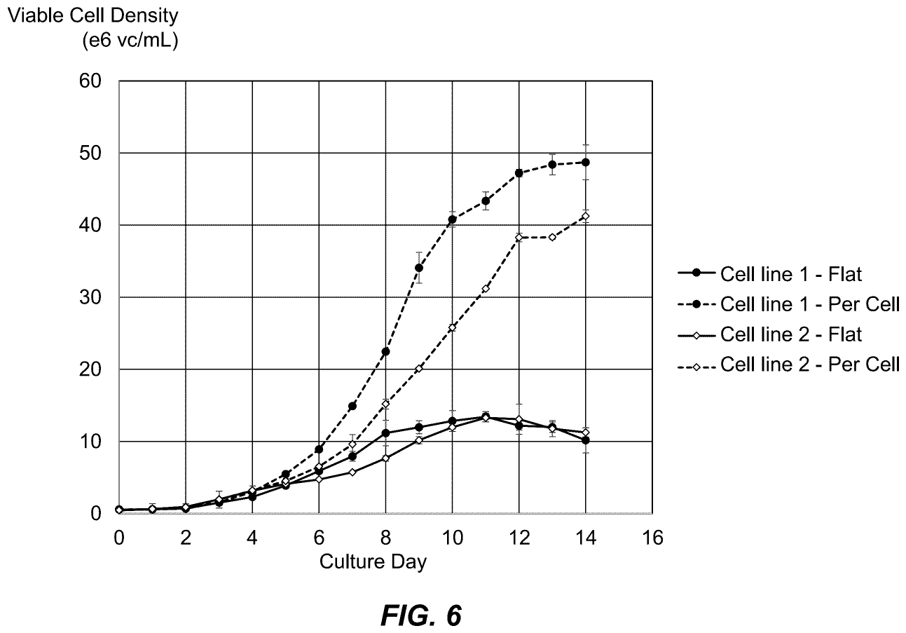

FIG. 6 is a plot of viable cell density for the two cell lines of the volume-based fed-batch bioreactor run and the cell-based fed-batch bioreactor run presented in FIG. 5. As illustrated in FIG. 5, both cell lines have a decrease in viability early in the process (day 2). As illustrated in FIG. 6, with the weight-based approach, both cell lines barely grow due to overfeeding and osmotic concentration accumulation. With the per-cell feeding schedule, growth recovers due to appropriate feeding and osmolarity in culture.

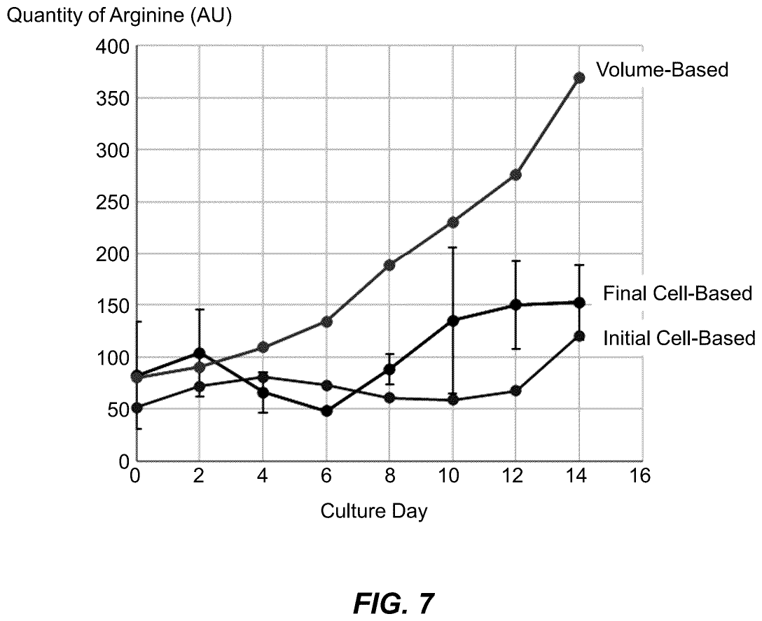

FIGS. 7 through 16 are plots of relative concentration, based on an internal standard, of a number of metabolites in the bioreactor over time. The metabolite concentration is dependent upon feed rate (amount of feed added per day) along with cellular consumption. When the feed is properly balanced and added at a rate that matches the cellular consumption, the net effect is that the concentration remains the same throughout the process. Generally, when a volume-based feed strategy was used, overfeeding and accumulation of metabolites was typical. This caused poor bioreactors performance. With the per-cell feed strategy, the metabolite levels remain largely stable and performance is improved.

FIG. 7 is a plot of relative concentration of arginine in the bioreactor during a volume-based fed-batch bioreactor run as known in the art, a first iteration of a cell-based fed-batch bioreactor run according to aspects of the present disclosure, and a final iteration of a cell-based fed-batch bioreactor run according to aspects of the present disclosure.

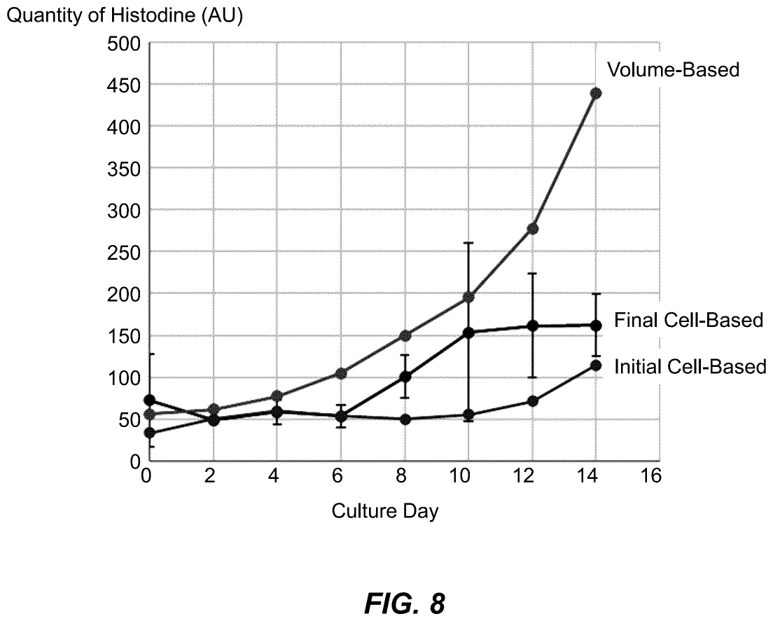

FIG. 8 is a plot of relative concentration of histidine in the bioreactor during a volume-based fed-batch bioreactor run as known in the art, a first iteration of a cell-based fed-batch bioreactor run according to aspects of the present disclosure, and a final iteration of a cell-based fed-batch bioreactor run according to aspects of the present disclosure.

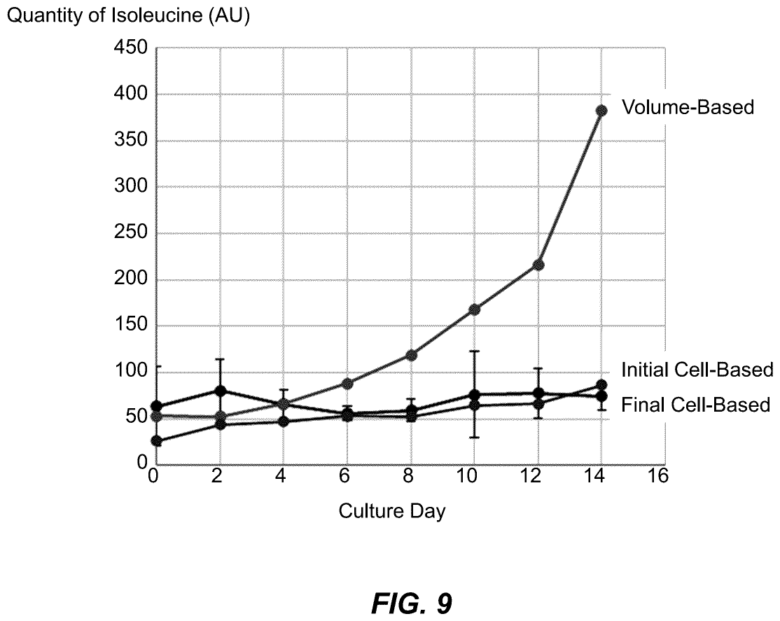

FIG. 9 is a plot of relative concentration of isoleucine in the bioreactor during a volume-based fed-batch bioreactor run as known in the art, a first iteration of a cell-based fed-batch bioreactor run according to aspects of the present disclosure, and a final iteration of a cell-based fed-batch bioreactor run according to aspects of the present disclosure.

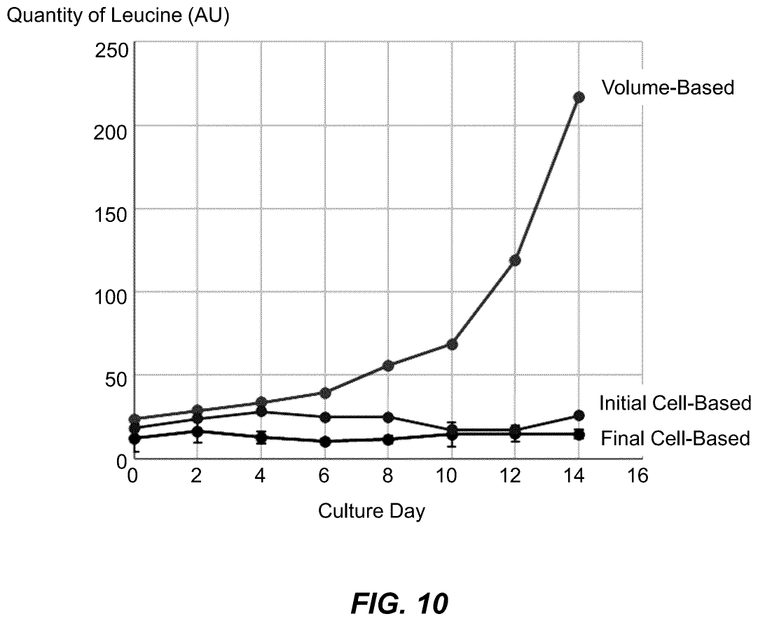

FIG. 10 is a plot of relative concentration of leucine in the bioreactor during a volume-based fed-batch bioreactor run as known in the art, a first iteration of a cell-based fed-batch bioreactor run according to aspects of the present disclosure, and a final iteration of a cell-based fed-batch bioreactor run according to aspects of the present disclosure.

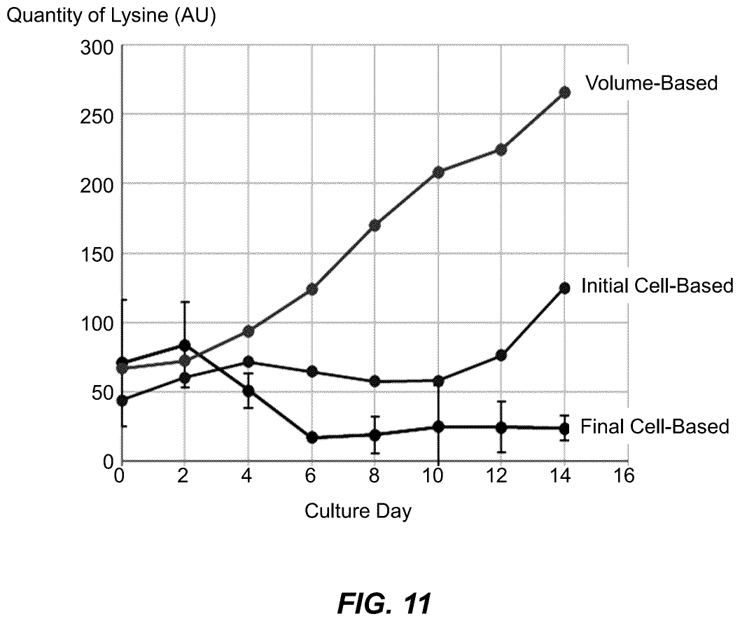

FIG. 11 is a plot of relative concentration of lysine in the bioreactor during a volume-based fed-batch bioreactor run as known in the art, a first iteration of a cell-based fed-batch bioreactor run according to aspects of the present disclosure and a final iteration of a cell-based fed-batch bioreactor run according to aspects of the present disclosure.

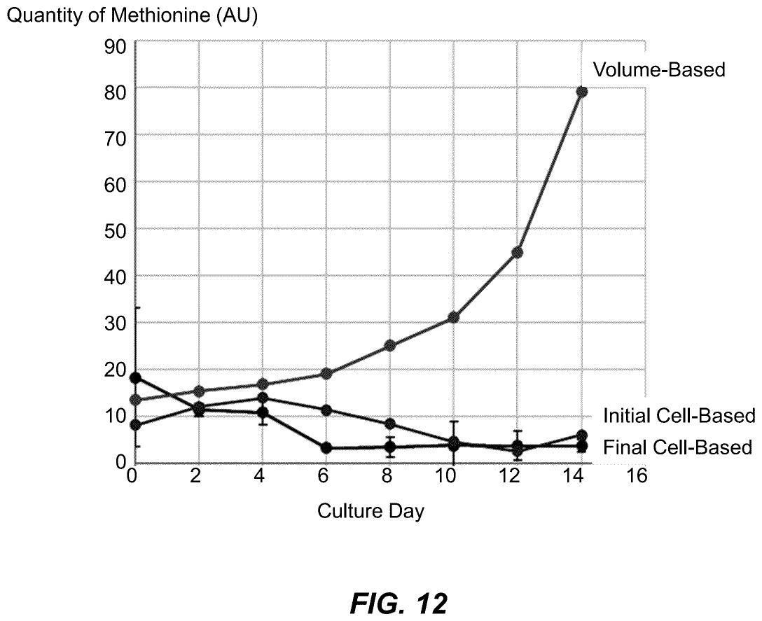

FIG. 12 is a plot of relative concentration of methionine in the bioreactor during a volume-based fed-batch bioreactor run as known in the art, a first iteration of a cell-based fed-batch bioreactor run according to aspects of the present disclosure and a final iteration of a cell-based fed-batch bioreactor run according to aspects of the present disclosure.

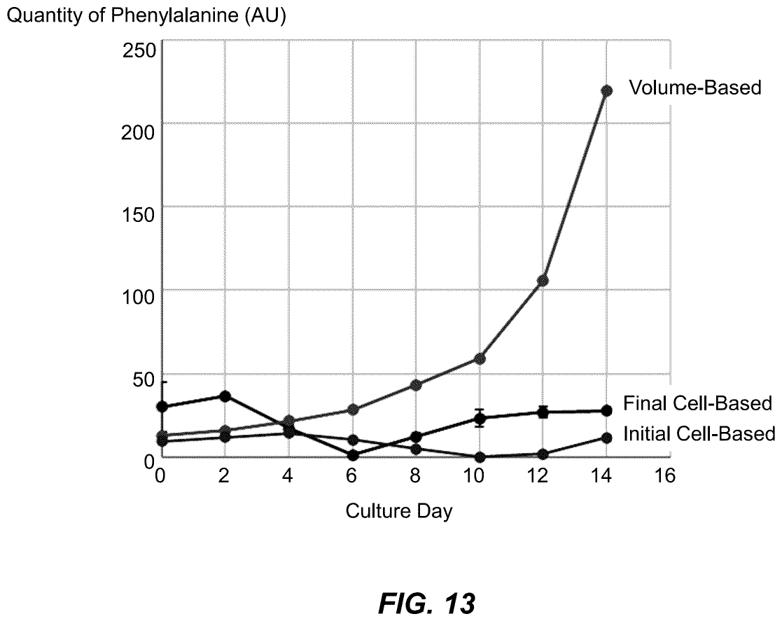

FIG. 13 is a plot of relative concentration of phenylalanine in the bioreactor during a volume-based fed-batch bioreactor run as known in the art, a first iteration of a cell-based fed-batch bioreactor run according to aspects of the present disclosure and a final iteration of a cell-based fed-batch bioreactor run according to aspects of the present disclosure.

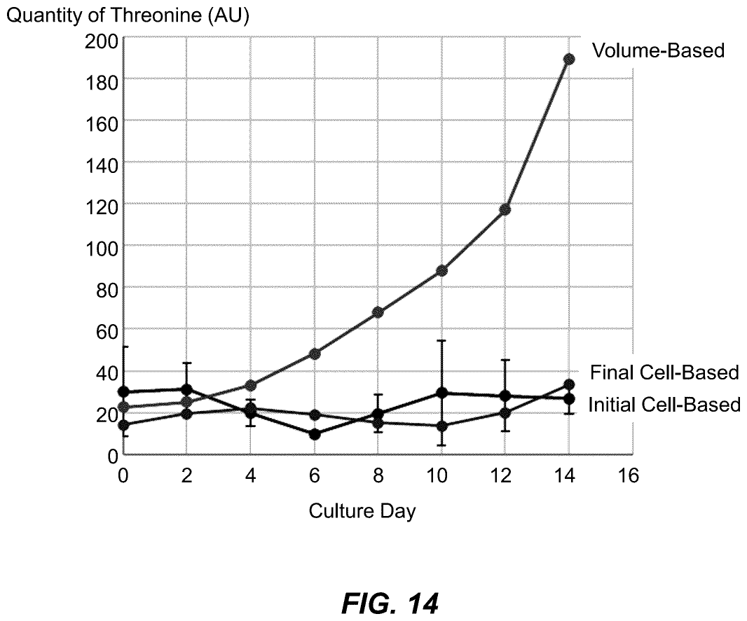

FIG. 14 is a plot of relative concentration of threonine in the bioreactor during a volume-based fed-batch bioreactor run as known in the art, a first iteration of a cell-based fed-batch bioreactor run according to aspects of the present disclosure and a final iteration of a cell-based fed-batch bioreactor run according to aspects of the present disclosure.

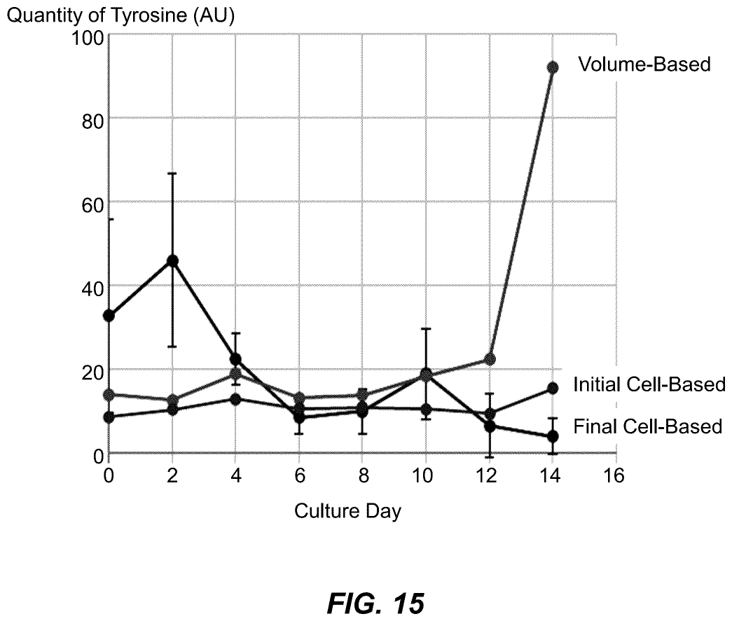

FIG. 15 is a plot of relative concentration of tyrosine in the bioreactor during a volume-based fed-batch bioreactor run as known in the art, a first iteration of a cell-based fed-batch bioreactor run according to aspects of the present disclosure and a final iteration of a cell-based fed-batch bioreactor run according to aspects of the present disclosure.

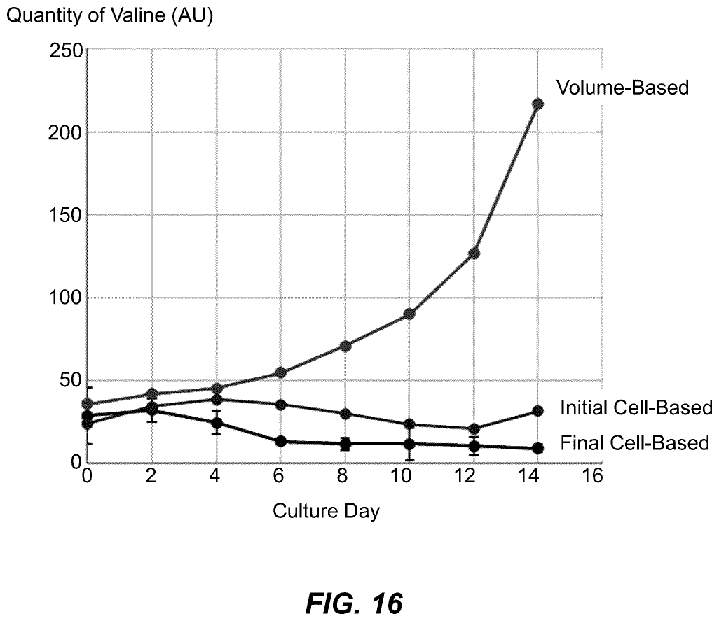

FIG. 16 is a plot of relative concentration of valine in the bioreactor during a volume-based fed-batch bioreactor run as known in the art, a first iteration of a cell-based fed-batch bioreactor run according to aspects of the present disclosure and a final iteration of a cell-based fed-batch bioreactor run according to aspects of the present disclosure.

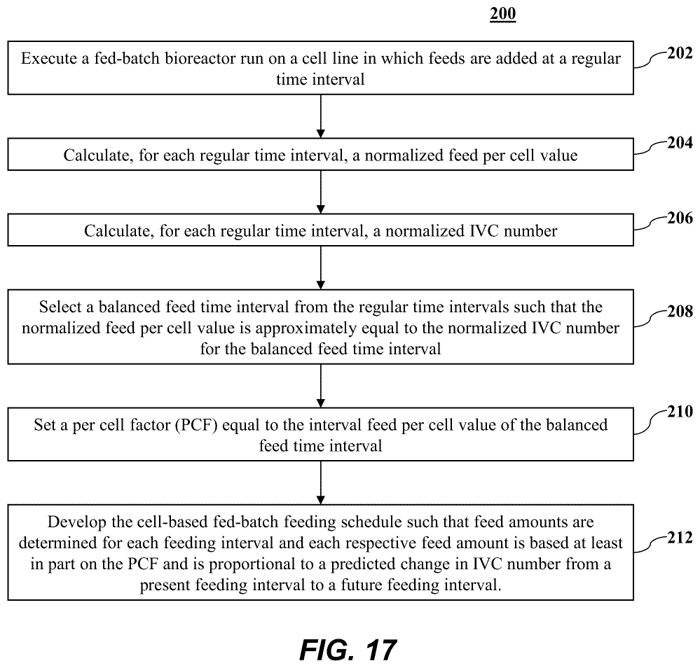

FIG. 17 is a flow diagram illustrating steps of an example method 200 for developing a cell-based fed-batch feeding strategy according to aspects of the present disclosure. At step 202, a fed-batch bioreactor run can be executed on a cell line. During the fed-batch bioreactor run, feeds can be added at a regular time interval. At step 204, a normal feed per cell value can be calculated for each regular time interval. At step 206, a normalized IVC number can be calculated for each regular time interval. At step 208, a balanced feed time can be selected from the regular time intervals such that the normalized feed per cell value is approximately equal to the normalized IVC number for the balanced feed time interval. At step 210, a PCF can be set equal to the interval feed per cell value of the balanced feed time interval. At step 212, the cell-based fed-batch feeding schedule can be developed such that feed amounts are determined for each feeding interval and each respective feed amount is based at least in part on the PCF and is proportional to a predicted change in IVC number from a present feeding interval to a future feeding interval.

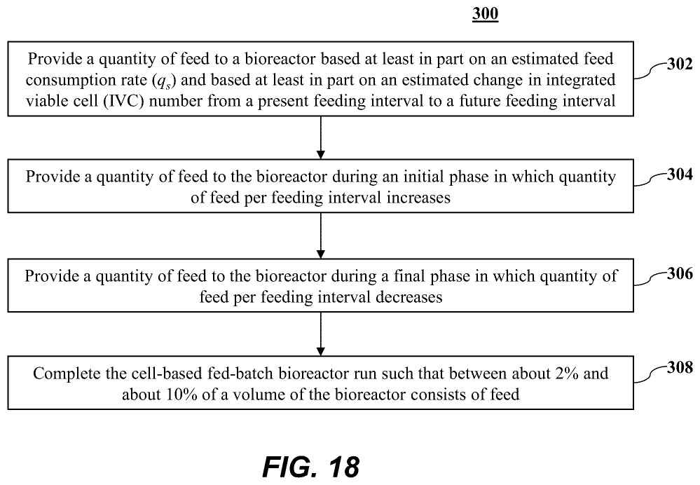

FIG. 18 is a flow diagram illustrating steps of an example method 300 for executing a cell-based fed-batch bioreactor run according to aspects of the present disclosure. At step 302, a quantity of feed is provided to a bioreactor based at least in part on an estimated feed consumption rate (qₛ) and based at least in part on an estimated change in IVC number from a present feeding interval to a future feeding interval. At step 304, a quantity of feed can be provided to the bioreactor during an initial phase in which quantity of feed per feeding interval increases. At step 306, a quantity of feed can be provided to the bioreactor during a final phase in which quantity of feed per feeding interval decreases. At step 308, the cell-based fed-batch bioreactor run can be completed such that between about 20% and about 50% of a volume of the bioreactor consists of feed. In the provided examples, complex feed accounts for about 25-35% of the total volume of a reactor at the end of the run. The range is expanded to 20% to 50% to account for different potential feed scenarios: Basal+Inoculum˜50%; Complex feed ˜25-35%; Glucose Feed ˜10-15%; and High PH Feed (˜2-5%).

Methods of executing a cell-based fed-batch bioreactor run and developing a cell-based fed-batch feeding schedule can be assisted by computer automation. Methods can be semi-automated or fully automated and may be organism agnostic. An external (to the bioreactor) system can include instructions to execute method steps for executing the cell-based fed-batch bioreactor run and developing a cell-based fed-batch feeding schedule.

Various analytical devices may be used in the present invention. The analytical devices may include any instrument or process that can detect and/or quantify a number of viable cells, component of a nutrient feed (e.g., an amino acid), and/or other substituents of cell culture media (e.g., a vitamin, a mineral, an ion, sugar, etc.). The analytical device may be an apparatus for performing gas chromatography, HPLC, cation exchange chromatography, anion exchange chromatography, size exclusion chromatography, an enzyme-catalyzed assay, and/or a chemical reaction assay.

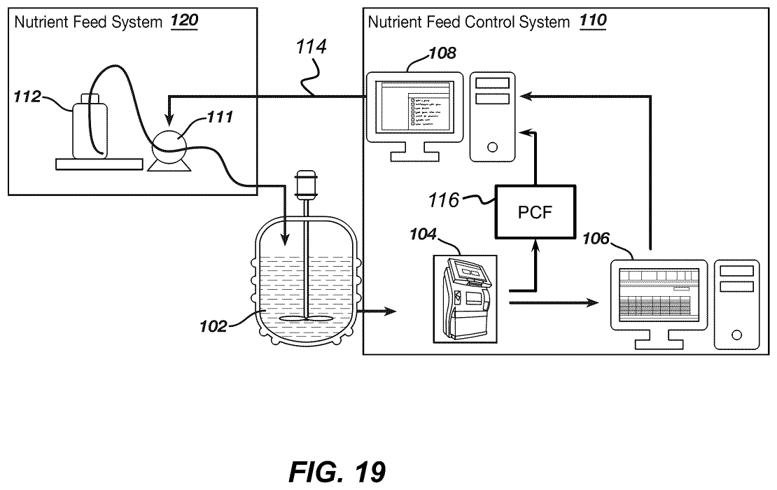

FIG. 19 is a schematic diagram of an example environment that may be used to implement one or more embodiments of the present disclosure. The environment includes a nutrient feed system 120, a production reactor 102, and a nutrient feed control system 110. The environment includes a feedback loop 114 from the nutrient feed control system 110 to a nutrient feed system 120 that can be used to regulate feed from the nutrient feed system 120 to the production reactor 102 during a cell-based fed-batch bioreactor run. The environment may also be used to develop a cell-based fed-batch feeding schedule.

The production reactor 102 may include cell culture (e.g. mammalian cell culture). The production reactor 102 may be a bioreactor, a cell culture reactor or a sample bioreactor. The production reactor 102 may be at least one of the following: a well plate, a shake flask, a bench top vessel, a cell bag and rocker, a single-use bioreactor and a commercial scale (e.g., 15 kL) stainless steel reactor. Reaction sample may be withdrawn from the production reactor 102, and sent to a nutrient feed control system 110.

The nutrient feed control system 110 may include a viable cell density measurement system 104 that determines density of viable cells in the production reactor 102. The viable cell density measurement may be performed either offline or online. The nutrient feed control system 110 may also include a future viable cell density prediction system 106 that receives viable cell density measurement from the viable cell density measurement system 104, and performs viable cell density number prediction. The nutrient feed control system 110 can include a PCF system 116. The PCF system 116 may simply provide a PCF value for a given feed interval, and/or the PCF system 116 may calculate a PCF value (e.g. single PCF value, average PCF value, and/or adjusted PCF value) as disclosed elsewhere herein. The nutrient feed control system 110 may include a feed calculation system 108 that may use the predicted viable cell density from the future viable cell density prediction system 106 and the PCF value from the PCF system 116 to calculate an amount of feed to add. The nutrient feed control system 110 may then send an instruction to the nutrient feed system 120 to provide feed according to the calculation performed by the feed calculation system 108. In one embodiment, processes performed by the viable cell density measurement system 104, the future viable cell density prediction system 106, the PCF system 116, and/or the feed calculation system 108 may be completed by one or more processors.

The nutrient feed system 120 may include a pump 111 which feeds the correct amount of feed from a feed source 112 to the production reactor 102.

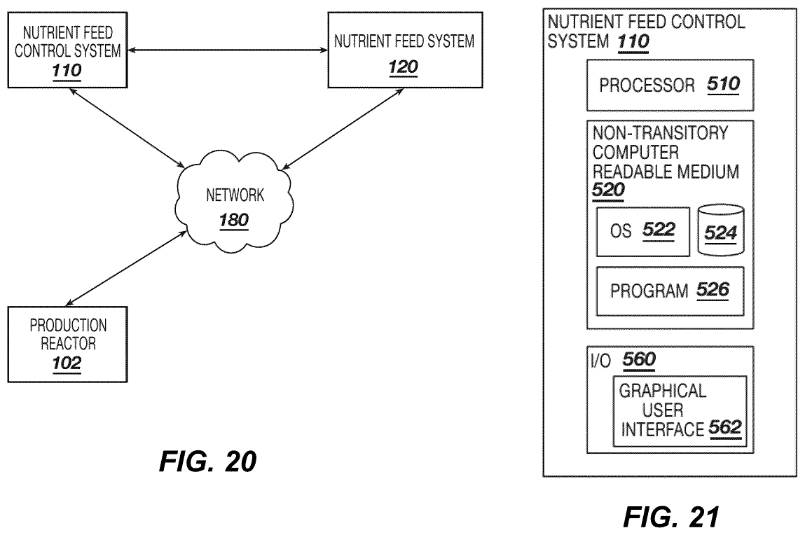

FIG. 20 illustrates schematic diagram of an example environment that may be used to implement one or more embodiments of the present disclosure. The nutrient feed control system 110 may communicate with the production reactor 102 and the nutrient feed system 120 over a network 180. The nutrient feed control system 110 may direct the nutrient feed system 120 to feed one or more nutrients to the production reactor 102.

In some embodiments, steps of an example method are performed by one or more automated devices. The terms “automatic”, “automatically”, or “automated” describe one or more mechanical devices that perform one or more tasks without any human intervention or action, except for any human intervention or action necessary to initially prepare the device or devices for task performance; or as may be required to maintain automatic operation of the device or devices. A “mechanical device” that performs one or more tasks automatically may, optionally, include a computer and the necessary instructions (code) therein to process collected data which may be used therein for decision making purposes to control and direct performance of the device or devices, such as in controlling the timing, duration, frequency, kind, and/or character of tasks to be performed.

In various embodiments, “off-line” analysis refers to permanently removing a sample from the production process and analyzing the sample at a later point in time such that the data analysis does not convey real-time or near real-time information about in-process conditions. In some embodiments, one or more analytical devices are used off-line.

In one embodiment, an analytical device (or a sensor-portion connected thereto) may be introduced directly into a bioreactor or purification unit, or the device or sensor-portion may be separated from the bioreactor or purification unit by an appropriate barrier or membrane.

In some embodiments, the analytical device may be a kit, e.g., a test strip, which can be placed in contact with the sample to give rapid determination of the cellular concentration. In some embodiment, the kit may comprise a substrate which produces a chemical and/or enzyme-linked reaction to produce a detectable signal in the presence of a surrogate marker, or a specific concentration of a surrogate marker. The detectable signal may include, e.g., a colorimetric change or other visual signal. In some embodiments, the analytical device may be a disposable analytical device, e.g., a disposable test strip. Such kits may be useful because of their ease of operation and their reduced costs relative to other larger, more complicated analytical devices. Such kits may also be useful during small scale cell culture propagation to determine that optimal health and productivity of the culture.

FIG. 21 is a block diagram of the nutrient feed control system 110 according to one aspect of the disclosed technology. The nutrient feed control system 110 may include one or more processors 510. The processes performed by the viable cell density measurement system 104, the future viable cell density prediction system 106, the PCF system 116, and/or the feed calculation system 108 may be completed by one or more processors 510.

The processor 510 may include one or more of a microprocessor, microcontroller, digital signal processor, co-processor or the like or combinations thereof capable of executing stored instructions and operating upon stored data. The processor 510 may be one or more known processing devices, such as a microprocessor from the Pentium™ family manufactured by Intel™ or the Turion™ family manufactured by AMD™. The processor 510 may constitute a single core or multiple core processor that executes parallel processes simultaneously. For example, the processor 510 may be a single core processor that is configured with virtual processing technologies. In certain embodiments, the processor 510 may use logical processors to simultaneously execute and control multiple processes. The processor 510 may implement virtual machine technologies, or other similar known technologies to provide the ability to execute, control, run, manipulate, store, etc. multiple software processes, applications, programs, etc. A person skilled in the pertinent art understands that other types of processor arrangements could be implemented that provide for the capabilities disclosed herein.

A non-transitory computer readable medium 520 may include, in some implementations, one or more suitable types of memory (e.g., such as volatile or non-volatile memory, random access memory (RAM), read only memory (ROM), programmable read-only memory (PROM), erasable programmable read-only memory (EPROM), electrically erasable programmable read-only memory (EEPROM), magnetic disks, optical disks, floppy disks, hard disks, removable cartridges, flash memory, a redundant array of independent disks (RAID), and the like), for storing files including an operating system 522, application programs (including, for example, a web browser application, a widget or gadget engine, and or other applications, as necessary), executable instructions and data. In one embodiment, the processing techniques described herein are implemented as a combination of executable instructions and data within the non-transitory computer readable medium 520. The non-transitory computer readable medium 520 may include one or more memory devices that store data and instructions used to perform one or more features of the disclosed embodiments. The non-transitory computer readable medium 520 may also include any combination of one or more databases controlled by memory controller devices (e.g., server(s), etc.) or software, such as document management systems, Microsoft™ SQL databases, SharePoint™ databases, Oracle™ databases, Sybase™ databases, or other relational or non-relational databases. The non-transitory computer readable medium 520 may include software components that, when executed by the processor 510, perform one or more processes consistent with the disclosed embodiments. In some embodiments, the non-transitory computer readable medium 520 may include a database 524 to perform one or more of the processes and functionalities associated with the disclosed embodiments. The non-transitory computer readable medium 520 may include one or more programs 526 to perform one or more functions of the disclosed embodiments. Moreover, the processor 510 may execute one or more programs 526 located remotely from the system 110. For example, the system 110 may access one or more remote programs 526, that, when executed, perform functions related to disclosed embodiments.

The system 110 may also include one or more I/O devices 560 that may comprise one or more interfaces for receiving signals or input from devices and providing signals or output to one or more devices that allow data to be received and/or transmitted by the system 110. For example, the system 110 may include interface components, which may provide interfaces to one or more input devices, such as one or more keyboards, mouse devices, touch screens, track pads, trackballs, scroll wheels, digital cameras, microphones, sensors, and the like, that enable the system 110 to receive data from one or more users. The system 110 may include a display, a screen, a touchpad, or the like for displaying images, videos, data, or other information. The I/O devices 560 may include the graphical user interface 562.

In exemplary embodiments of the disclosed technology, the system 110 may include any number of hardware and/or software applications that are executed to facilitate any of the operations. The one or more I/O interfaces 560 may be utilized to receive or collect data and/or user instructions from a wide variety of input devices. Received data may be processed by one or more computer processors as desired in various implementations of the disclosed technology and/or stored in one or more memory devices.

The networks 180 may include a network of interconnected computing devices more commonly referred to as the internet. The network 180 may be of any suitable type, including individual connections via the internet such as cellular or WiFi networks. In some embodiments, the network 180 may connect terminals, services, and mobile devices using direct connections such as radio-frequency identification (RFID), near-field communication (NFC), Bluetooth™ low-energy Bluetooth™ (BLE), WiFi™, ZigBee™, ambient backscatter communications (ABC) protocols, USB, WAN, or LAN. Because the information transmitted may be personal or confidential, security concerns may dictate one or more of these types of connections be encrypted or otherwise secured. In some embodiments, however, the information being transmitted may be less personal, and therefore the network connections may be selected for convenience over security. The network 180 may comprise any type of computer networking arrangement used to exchange data. For example, the network 180 may be the Internet, a private data network, virtual private network using a public network, and/or other suitable connection(s) that enables components in system environment to send and receive information between the components of system 100. The network 180 may also include a public switched telephone network (“PSTN”) and/or a wireless network. The network 180 may also include local network that comprises any type of computer networking arrangement used to exchange data in a localized area, such as WiFi, Bluetooth™ Ethernet, and other suitable network connections that enable components of system environment to interact with one another.

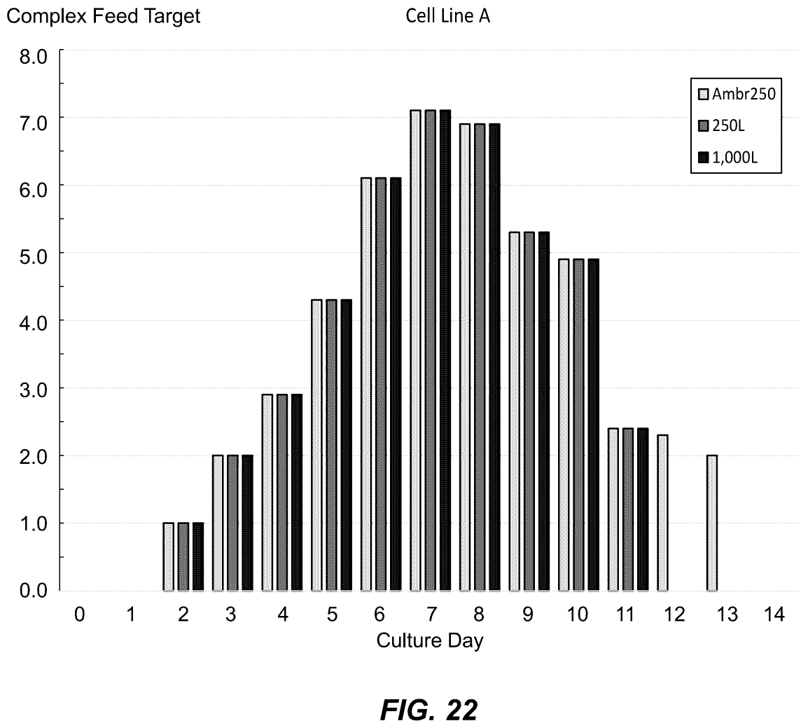

FIGS. 22 through 26 relate to large-scale production for two exemplary molecules monoclonal antibody 1 (MAB1) and BsAb1. at 0.25 L, 250 L, and 1,000 L scales. The complex feed targets were developed during reduced-scale runs using the predictive cell-based algorithm and designed to maximize productivity while minimizing the accumulation of inhibitory waste by-products (e.g., lactate and ammonium). The large-scale production of MAB1 and BsAb1 demonstrated that complex feed targets developed during reduced-scale studies using the predictive cell-based algorithm were transferrable across scales.

FIG. 22 is a bar chart showing daily complex feed targets at three different scales for production of MAB1. Culture days 2 through 11 include bars for Ambr250 (0.25 L scale), 250 L, and 1,000 L processes ordered from left to right for each day. There are no feed targets for culture days 12 and 13 for 250 L and 1,000 L processes as the processes were stopped after day 12 based on Ambr250 data.

The daily complex feed targets developed for the production bioreactor process for MAB1 were predicted during an Ambr250 experiment using the cell-based algorithm. The algorithm provided daily feed targets based on the current biomass in the reactor with ranges of ±10% to study the impact of the slight misfeeds that may happen during manufacturing. The process performance is evaluated with respect to titer, osmolality, lactate and ammonium concentration as a function of the range of complex feed delivered. The daily feed target for each duplicate condition in the study is then averaged and that value is the recommended target for use in future process development studies. The predicted complex feed targets from the Ambr250 (0.25 L) development study are then applied to both a pilot scale (250 L) and larger-scale process (1,000 L). Both the 250 L and 1,000 L processes used the same complex feed targets initially identified in the early development Ambr250 study.

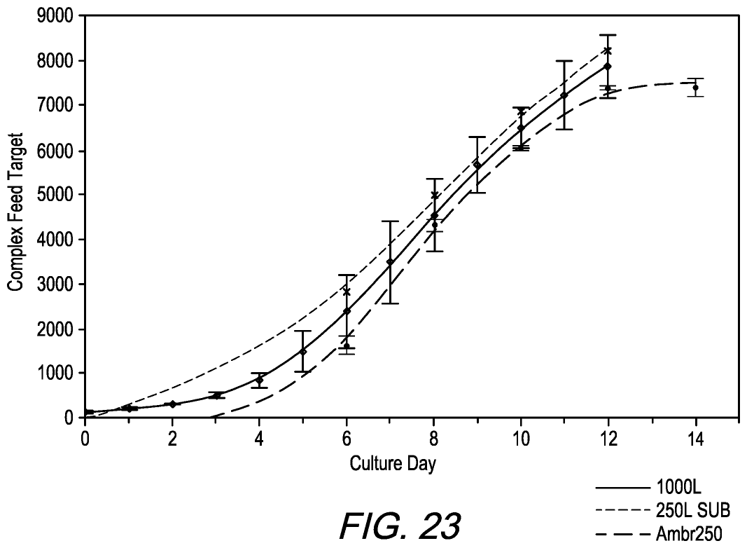

FIG. 23 is a plot of titer values for three different scales for production of MAB1.

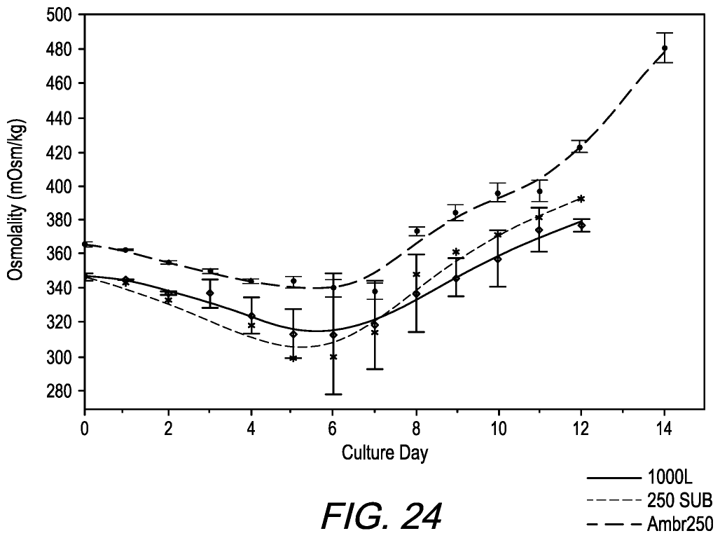

FIG. 24 is a plot of daily measured osmolality for three different scales for production of MAB1.

In the plots of FIGS. 23 and 24, Ambr250 (0.25 L) data is shown in long light gray dashes, 250 L data is shown in short dark gray dashes, and 1,000 L data is shown in a solid black line with open diamonds.

Upon scale up, both titer (FIG. 23) and osmolality (FIG. 24) trended in line with the original algorithm-predicted process and are within experimental and biological variability. This indicates that the predicted complex feed targets are appropriate across scales. An offset in initial osmolality (FIG. 24) is likely due to differences in split ratios at inoculation.

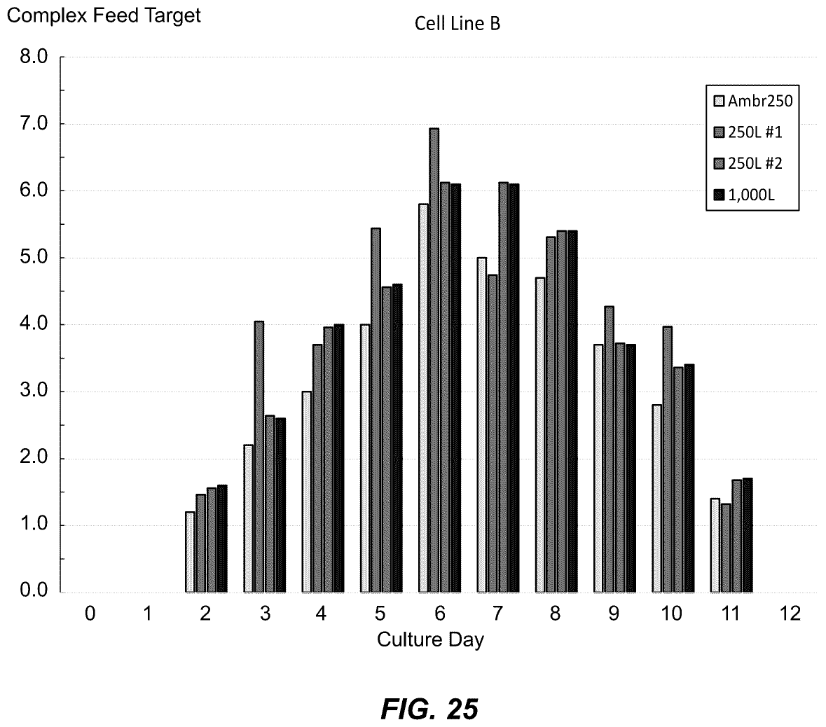

FIG. 25 is a bar chart showing daily complex feed targets at 0.25 L, 250 L, and 1,000 L scales for production of BsAb1. Culture days 2 through 11 include bars for Ambr250 (0.25 L scale), 250 L #1, 250 L #2, and 1,000 L processes ordered from left to right for each day. The daily complex feed targets developed for the production bioreactor process for BsAb1 were predicted during an Ambr250 and two, 250 L SUB experiments using the cell-based glucose algorithm. The algorithm provided daily complex feed targets based on the current biomass in the reactor. The Ambr250 studied also included targets with ranges of #10% to study the impact of the slight misfeeds that may happen during manufacturing. Algorithm predicted targets were evaluated for three separate processes and the outputs from the third experiment (250 L #2, FIG. 25) were chosen as the complex feed targets to transfer for large scale production.

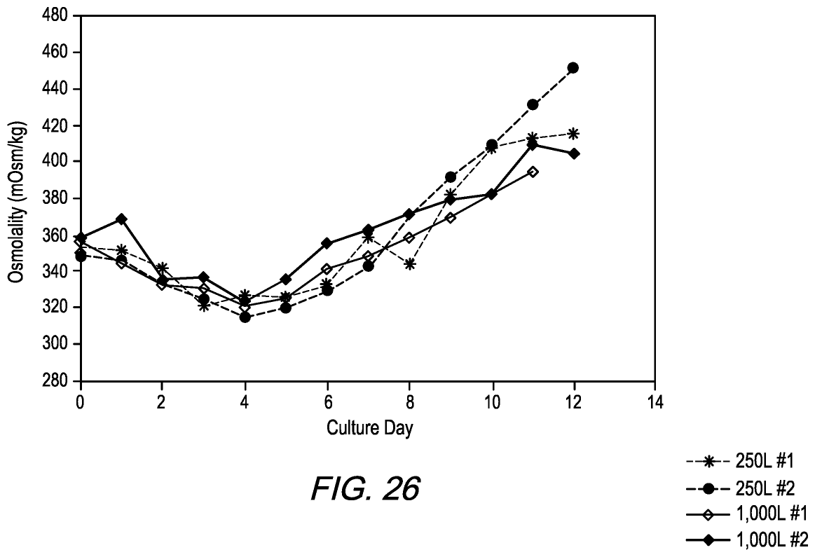

FIG. 26 a plot of daily measured osmolality for three different scales for production of BsAb1. The predicted complex feed targets from the second pilot scale experiment (250 L #2, FIG. 25) were then applied to the large scale process (1,000 L). The pilot scale study was executed first to generate material for additional development studies. Upon scale up, both titer (data not shown) and osmolality (FIG. 26) trended in line with the original algorithm-predicted process and are within experimental and biological variability. This indicates that the predicted complex feed targets are appropriate across scales.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

Certain implementations of the disclosed technology are described above with reference to block and flow diagrams of systems and methods and/or computer program products according to example implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, or may not necessarily need to be performed at all, according to some implementations of the disclosed technology.

These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks.

Implementations of the disclosed technology may provide for a computer program product, comprising a computer-usable medium having a computer-readable program code or program instructions embodied therein, said computer-readable program code adapted to be executed to implement one or more functions specified in the flow diagram block or blocks. The computer program instructions may also be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

1. A method of executing a cell-based fed-batch bioreactor run, the method comprising:
providing a quantity of feed to a bioreactor based at least in part on an estimated feed consumption rate (qₛ) and based at least in part on a predicted change in integrated viable cell (IVC) number from a present feeding interval to a future feeding interval.

2. The method of claim 1, further comprising:
predicting the change in IVC number as follows:
determining an IVC number of a present feeding interval,
estimating a growth rate based on the viable cell density of the present feeding interval and the viable cell density of a previous feeding interval,
predicting an IVC number for a following feeding interval based at least in part on the estimated growth rate, and
setting the change in IVC equal to the IVC number for the following feeding interval minus the IVC number for the present feeding interval.

3. The method of claim 1, wherein providing the quantity of feed to a bioreactor is based at least in part on a Per Cell Factor (PCF), and the PCF is proportionate to the estimated feed consumption rate (qₛ).

4. The method of claim 2, wherein the PCF varies over time during the cell-based fed-batch bioreactor run.

5. The method of claim 2, wherein the PCF does not increase over time during the cell-based fed-batch bioreactor run.

6. The method of claim 2,
wherein the PCF is approximately equal to 0.002 g/cell×day for culture days 2 through 3 of the cell-based fed-batch bioreactor run,
wherein the PCF is approximately equal to 0.00175 g/cell×day for culture days 4 through 6 of the cell-based fed-batch bioreactor run,
wherein the PCF is approximately equal to 0.0015 g/cell×day for culture days 7 through 8 of the cell-based fed-batch bioreactor run,
wherein the PCF is approximately equal to 0.0012 g/cell×day for culture days 9 through 10 of the cell-based fed-batch bioreactor run, and
wherein the PCF is approximately equal to 0.0007 g/cell×day for culture days 11 through 13 of the cell-based fed-batch bioreactor run.

7. The method of claim 1, further comprising:
providing the quantity of feed to the bioreactor such that for each feed interval during the fed-bath bioreactor run, an amount of metabolite added to the bioreactor during each respective feed interval run is approximately equal to an amount of metabolite consumed within the bioreactor during each respective feed interval.

8. The method of claim 1, further comprising:
completing the cell-based fed-batch bioreactor run such that between about 20% and about 50% of a volume of the bioreactor consists of feed.

9. The method of claim 1, wherein providing the quantity of feed comprises providing a complex nutrient feed comprising at least one of: alanine, arginine, asparagine, aspartic acid, cysteine, cystine, glutamic acid, glutamine, glycine, histidine, isoleucine, leucine, lysine, methionine, phenylalanine, proline, hydroxy-L-proline, serine, threonine, tryptophan, tyrosine, valine, amino acid di- and tri-peptides, B vitamins, carbohydrates, lipids, antioxidants, growth factors and trace elements.

10. The method of claim 1, further comprising:
providing the quantity of feed to the bioreactor in an initial stage comprising a first plurality of feeding intervals and a subsequent final stage comprising a second plurality of feeding intervals,
wherein the quantity of feed increases from one feeding interval to a subsequent feeding interval for each of the first plurality of feeding intervals of the initial stage, and
wherein the quantity of feed decreases, or remains constant, from one feeding interval to a subsequent feeding interval for each of the second plurality of feeding intervals of the final stage.

11. The method of claim 1, further comprising:
completing the cell-based fed-batch bioreactor run such that a viable cell density in the bioreactor is approximately 3 to approximately 5 times a resulting viable cell density of a volume-based fed-batch bioreactor run executed under identical conditions, but for feeding strategy, as the cell-based fed-batch bioreactor run.

12. The method of claim 1, wherein the estimated feed consumption rate (qₛ) and IVC are each based on a reduced-scale study of the cell-based fed-batch bioreactor run.

13. The method of claim 12, wherein the estimated feed consumption rate (qₛ) and IVC are each based on about a 0.25 L study of the cell-based fed-batch bioreactor run.

14. A method of developing a cell-based fed-batch feeding schedule, the method comprising:
executing a fed-batch bioreactor run on a cell line in which feeds are added at a regular time interval;
calculating, for each regular time interval, a normalized feed per cell value as follows:
for each regular time interval, calculating an interval feed per cell value that is a feed quantity fed at the respective time interval divided by a change in an integrated viable cell (IVC) number from a previous time interval to the respective time interval,
determining a maximum interval feed per cell value of the calculated interval feed per cell values, and
for each regular time interval, setting the normalized feed per cell value equal to the interval feed per cell value for the respective time interval divided by the maximum interval feed per cell value;
calculating, for each regular time interval, a normalized IVC number as follows:
for each regular time interval, estimating a daily IVC number based at least in part on an estimated IVC number of the previous time interval and a change in viable cells from the previous time interval to the respective time interval,
determining a maximum daily IVC number of the estimated daily IVC numbers, and
for each regular time interval, setting the normalized IVC number to the estimated daily IVC number divided by the maximum daily IVC number;
selecting a balanced feed time interval from the regular time intervals such that the normalized feed per cell value is approximately equal to the normalized IVC number for the balanced feed time interval;
setting a per cell factor (PCF) equal to the interval feed per cell value of the balanced feed time interval; and
developing the cell-based fed-batch feeding schedule such that feed amounts are determined for each feeding interval and each respective feed amount is based at least in part on the PCF and is proportional to a predicted change in IVC number from a present feeding interval to a future feeding interval.

15. The method of claim 14, further comprising:
executing at least four fed-batch bioreactor runs on the cell line in which feeds are added at the regular time interval;
for each regular time interval of each of the at least four fed-batch bioreactor runs, calculating the normalized feed per cell value;
for each regular time interval of each of the fed-batch bioreactor runs, calculating the normalized IVC number;
for each fed-batch bioreactor run, selecting the balanced feed time interval;
for each fed-batch bioreactor run, setting the PCF equal to the respective interval feed per cell value of the respective balanced feed time interval;
determining an average PCF that is an average of the PCFs of the at least four fed-batch bioreactor runs; and
developing the cell-based fed-batch feeding schedule such that feed amounts are determined for each feeding interval and each respective feed amount is equal to the average PCF multiplied by the predicted change in IVC number.

16. The method of claim 14, further comprising:
executing a first cell-based fed-batch bioreactor run according to the fed-batch feeding schedule; and
adjusting the PCF such that you minimize the amount of feed added to a bioreactor without compromising growth, productivity and/or depleting essential nutrients.

17. The method of claim 14, further comprising:
executing a first cell-based fed-batch bioreactor run according to the fed-batch feeding schedule;
determining an updated PCF based at least in part on said PCF and based at least in part on the first cell-based fed-batch bioreactor run; and
executing a second cell-based fed-batch bioreactor run according to an updated fed-batch feeding schedule utilizing the updated PCF such that the second cell-based fed-batch bioreactor run utilizes a decreased total amount of feed added compared to the first cell-based fed-batch bioreactor run.

18. The method of claim 14, further comprising:
executing the first cell-based fed-batch bioreactor run according to the fed-batch feeding schedule; and
determining, based at least in part on said PCF and based at least in part on the first cell-based fed-batch bioreactor run, a variable PCF and a variable fed-batch feeding schedule.

19. The method of claim 18, wherein the variable PCF does not increase over time during the cell-based fed-batch bioreactor run.

20. The method of claim 18,
wherein the variable PCF is approximately equal to 0.002 g/cell×day for culture days 2 through 3 of the cell-based fed-batch bioreactor run,
wherein the variable PCF is approximately equal to 0.00175 g/cell×day for culture days 4 through 6 of the cell-based fed-batch bioreactor run,
wherein the variable PCF is approximately equal to 0.0015 g/cell×day for culture days 7 through 8 of the cell-based fed-batch bioreactor run,
wherein the variable PCF is approximately equal to 0.0012 g/cell×day for culture days 9 through 10 of the cell-based fed-batch bioreactor run, and
wherein the variable PCF is approximately equal to 0.0007 g/cell×day for culture days 11 through 13 of the cell-based fed-batch bioreactor run.

21. The method of claim 14, wherein the cell-based fed-batch feeding schedule is developed at a reduced scale to provide feed targets that are transferrable across scales.

22. The method of claim 21,
wherein the cell-based fed-batch feeding schedule is developed at about a 0.25 L scale, and
wherein the cell-based fed-batch feeding schedule is transferrable across fed-batch production processes utilizing about 250 liter (L) and about 1,000 L bioreactors respectively.

23. A nutrient feed control system comprising:
a viable cell density measurement system configured to determine density of viable cells in a production reactor;
a future viable cell density prediction system configured to receive the viable cell density measurement and configured to estimate a density of viable cells in a future feed time interval based at least in part on the viable cell density measurement;
a per cell factor (PCF) system configured to provide a PCF value; and
a feed calculation system configured to calculate an amount of feed to provide to the production reactor based at least in part on the density of viable cells in the future feed time interval and based at least in part on the PCF value.

24. The nutrient feed control system of claim 23, wherein the PCF system is further configured to:
determine a balanced feed time interval at which metabolites consumed in the bioreactor are approximately equal to metabolites provided to the bioreactor on during the balanced feed time interval,
determine a feed per cell value for the balanced feed time interval, and
determine the PCF value based at least in part on the feed per cell value for the balanced feed time interval.

25. The nutrient feed control system of claim 14, further comprising:
a processor; and
non-transitory computer-readable medium in communication with the processor with instructions thereon that when executed by the processor, cause the processor to calculate the amount of feed to provide to the production reactor.

26. The nutrient feed control system of claim 14, further comprising:
a processor; and
non-transitory computer-readable medium in communication with the processor with instructions thereon that when executed by the processor, cause the processor to determine the PCF value.

27. The nutrient feed control system of claim 23, wherein the feed calculation system is further configured to scale the amount of feed to provide to a larger production reactor of one or more larger scale fed-batch production processes.

28. The nutrient feed control system of claim 27, wherein the feed calculation system is further configured to scale the amount of feed to provide to the larger production reactor based on a 0.25 L scale process.

29. The nutrient feed control system of claim 23, wherein the feed calculation system is further configured to scale the amount of feed to provide to 250 liter (L) and a 1,000 L bioreactors during respective larger scale red-batch production processes.
